(19) United States
(12) Patent Application Publication
(10) Pub. No.: US 2025/0320447 A1
(43) Pub. Date: Oct. 16, 2025

(54) CENTRIFUGAL IMPACT-BASED BIOAEROSOL ENRICHMENT DEVICE, AND CELL EXPOSURE AND USE THEREOF

(71) Applicants: LIAONING TECHNICAL UNIVERSITY, Fuxin, Liaoning (CN); GUANGDONG UNIVERSITY OF TECHNOLOGY, Guangzhou, Guangdong (CN)

(72) Inventors: Ting ZHANG, Fuxin (CN); Taicheng AN, Guangzhou (CN); Guiying LI, Guangzhou (CN)

(73) Assignees: LIAONING TECHNICAL UNIVERSITY, Fuxin, Liaoning (CN); GUANGDONG UNIVERSITY OF TECHNOLOGY, Guangzhou, Guangdong (CN)

(21) Appl. No.: 18/873,291

(22) PCT Filed: Jun. 9, 2023
(86) PCT No.: PCT/CN2023/099515

§ 371 Date: Dec. 9, 2024

(30) Foreign Application Priority Data
Jun. 9, 2022 (CN) 202210645417.8

(51) Int. Cl. C12M 1/34; C12M 1/26; C12M 1/36; G01N 33/50

(52) U.S. Cl. CPC C12M 41/36; C12M 33/00; C12M 41/12; C12M 41/34; C12M 41/46; C12M 41/48; G01N 33/5014

(57) ABSTRACT

Disclosed are a centrifugal impact-based bioaerosol enrichment device, and cell exposure and use thereof. The device includes a bioaerosol enrichment device main unit and a cell exposure system; air sequentially passes through an enrichment chamber, a balance pool, and an exposure module of the enrichment device main unit; the cell exposure system collects and adjusts exposure data of bioaerosols in the air in real time; a magnetic levitation mechanism drives a cell exposure membrane box to rotate. The steps of real-time collection, intelligent adjustment, dynamic control and exposure are sequentially carried out to perform air bioaerosol enrichment and dynamic monitoring of cell exposure toxicity. The bioaerosol enrichment device, and the cell exposure and use thereof according to the present disclosure have simple and easy-to-perform methods, reliable data, and high accuracy; solve problems about enrichment and mobile monitoring of low concentration bioaerosols (including viruses, bacteria, fungi, and the like on particles) in the air; can be applied to crowd activity places such as transportation vehicles, livestock farms, biological laboratories, and residential areas; and have great application value.

### FIELD OF THE INVENTION

The present disclosure belongs to the technical field of bioaerosol monitoring, and discloses a bioaerosol enrichment device, and a cell exposure research method and use thereof, which can be applied to real-time monitoring, enrichment, and toxicity evaluation of bioaerosols in air.

### BACKGROUND OF THE INVENTION

In recent years, health hazards posed by indoor and outdoor air pollutants occur frequently, and the air pollution problem has become the focus of social attention. Aerosols, as harmful airborne pollutants, necessitate the development of rapid, accurate, and reliable detection methods. At present, a collection technology for bioaerosols in air mainly includes a liquid impact method, a solid impact method, a centrifugal sedimentation method, and a filter membrane method.

High and medium-flow collectors can be used to capture airborne pollutants, and can effectively enrich low-concentration aerosols in the air. Methods such as a suction sampling method, a filter membrane method, and an agar sampling method are commonly employed. However, these methods typically measure instantaneous concentrations, which leads to uncertainty. A method for collecting bioaerosols by using a vessel (for example, a sampling canister, a sampling tag, and a sampling bottle) is suitable for collecting pollution gas which have high concentration, high measurement sensitivity, are not easily adhered, or have stable chemical properties in a short period of time. The concentrations of biological pollutants collected by the above methods often cannot simply and quickly characterize a time weighted average concentration of bioaerosols in the air, or a cumulative concentration over a long period of time (such as a day, a night, a month, and a quarter).

As requirements for environmental biological pollution monitoring increase, existing environmental monitoring technologies of China continue to advance, including the use of a cell-based biosensor and other methods for monitoring pollutants in the environment. Researchers from the Ecological Environment Research Center of the Chinese Academy of Sciences have found that a signal of an ionized cell-based biosensor is enhanced in a specific environment, while a shape of an immobilized cell-based biosensor changes due to the influence of factors such as ion concentration in the air, device resistance, and time, resulting in a problem that a detection signal of low concentration benzene pollutants in the air is not obvious. These biological monitoring methods often lack necessary system optimization and have high uncertainty. In recent years, methods for testing toxicity of bioaerosols in air are gradually been developed. Previous researches focused on the toxicity and genotoxicity of a single pollutant to cells, and there are few methods for researching cell exposure of all-components bioaerosols in a real natural environment. Test results often fail to comprehensively reflect dynamics of bioaerosols in ambient air, or cytotoxicity and risks of exposure to real air pollutants.

The applicant of the present disclosure believes that replacing an impact type Anderson atmospheric bioaerosol sampler or a conventional membrane filter with a centrifugal impact-based enrichment chamber facilitates enrichment of microbial aerosols (such as pathogenic bacteria adhered to particles) on an elastic gel bin plate. A main reason lies in the porous and hygroscopic nature of a hydrogel material, which ensures that chemical compositions, morphological structures, and aerodynamic properties of the bioaerosols adhered to a bin plate are minimally affected by internal resistance in a device. The centrifugal impact-based enrichment method enables monitoring and exposure data to be closer to “in-situ” atmospheric bioaerosol concentration without significantly increasing detection costs.

A mobile centrifugal impact-based bioaerosol enrichment device disclosed in the present disclosure is compact in structure and convenient to operate. It is mobile, for example, carried on mobile devices such as an unmanned aerial vehicle and a mobile car, or worn on the human body, suitable for detecting concentration of biological pollutants in air in crowd activity places such as campuses, bathrooms, supermarkets, and laboratories, and providing basis data for developing control measures for pathogenic microorganisms propagated through an air medium. The present disclosure further provides a cell exposure method for bioaerosols, which facilitates in-situ detection of cell exposure toxicity of the bioaerosols and has broad application prospects.

### SUMMARY OF THE INVENTION

An objective of the present disclosure is to overcome in the foregoing prior art, discloses a mobile centrifugal impact-based enrichment device suitable for low concentration bioaerosols in air, and provides a method and use for conducting cell exposure research based on steps of real-time collection, intelligent adjustment, and dynamic control.

According to a mobile centrifugal impact-based bioaerosol enrichment device, and a cell exposure research method and use thereof in the present disclosure, the device includes an air bioaerosol enrichment device main unit and a cell-based biosensor system. The bioaerosol enrichment device main unit includes an enrichment chamber I with a grading function, a balance pool II, and an exposure module III, which can be achieved through the following technical solution:

The enrichment chamber I is of a multi-chamber structure. Chambers include porous gas distribution plates with bore diameters ranging from 0.001 to 50 mm, and include a heavy dust bin, a trace dust bin, a microbial enrichment bin, a sampling loop, and an automatic controller.

A wall surface of the enrichment chamber I may be made of a porous grid and a porous membrane tube. A material may be ceramics, polymer materials, titanium alloy, and the like, preferably, shape-memory titanium-nickel alloy, and a surface roughness Ra/Rz/Ry is greater than or equal to 0.8.

A wall surface of a trace dust bin of the enrichment chamber I is provided with a micro-groove structure with a depth ranging from 1 μm to 1 mm, including one or a combination of a wedge shape, a triangle, a needle shape, and hexagonal prism. A wireless sensor, including, a micron Particulate Matter (PM) concentration sensor, is buried at a top.

An ultraviolet-light-emitting diode (UV-LED) strip with an adjustable wavelength is arranged at a bottom of the heavy dust bin of the enrichment chamber I, and the adjustable wavelength ranges from 200 to 400 nm.

The microbial enrichment bin of the enrichment chamber I is of a porous membrane tube structure, and a wall surface is coated with gel, which facilitates trapping microbes in air, and maintaining microbial activity.

The porous membrane tube according to the present disclosure is a gel membrane tube including the following components in parts by weight, and a preferred hydrogel solution formula includes:
20 to 40 parts of polysulfone (PSU)
1 to 10 parts of polyvinylidene fluoride (PVDF)
7.5 to 50 parts of solvent
3.5 to 10 parts of pore-forming agent
0.1 to 3.5 parts of surfactant
0.001 to 1.5 parts of biological dye

A specific preparation method is as follows:
standing a hydrogel solution at a constant temperature, injecting the hydrogel solution into a spinneret, and drawing for 8 to 10 minutes to solidify and form in a water bath at 4 to 40° C. to obtain the porous membrane tube, preferably, a cyclone type membrane tube.

The balance pool II has a function of adjusting a concentration of bioaerosols in air. A filter mesh, an atomizer, and an electrostatic generator are mounted. A bottom end of the balance pool II is connected to a top end of the enrichment chamber I.

A size of the filter mesh of the balance pool II is greater than 0.01 mm, and a separating particle size ranges from 0.001 μm to 50 mm. The filter mesh may be made of polytetrafluoroethylene (PTFE), polyethersulfone (PES), polysulfone (PSU), polyacrylonitrile (PAN), cellulose acetate (CA), wood pulp filter paper, and stainless steel, and preferably, PTFE, but is not limited to these.

The atomizer of the balance pool II is a disc type atomizing droplet generator. A diameter of the disc does not exceed 100 mm, and a sauter mean diameter (SMD) of atomizing droplets ranges from 0.1 to 1 mm.

An output voltage of the electrostatic generator of the balance pool II may be adjusted between 2.5 and 80 KV. The electrostatic generator includes two-stage beehive electrostatic adsorption device, which may be combined for use with a negative ion generator, but is not limited this.

The balance pool II may be provided with a solenoid valve capable of adjusting opening and a starting speed to perform partitioned management on air with different bioaerosol concentrations and continuously or intermittently to distribute the air to a cell exposure membrane box of the exposure module III.

In some embodiments, the electrostatic generator of the balance pool II may be swapped with the filter mesh and the atomizer. A housing of the filter mesh may be externally connected to a biological reagent interface to adjust air inlet quality of the exposure module III. Temperature, humidity, PM concentration, and microbial concentration sensors and a global positioning system (GPS)/location-based service (LBS)/Beidou tracking locator may be mounted to input data to a data processing unit in real time.

The exposure module III is arranged below the enrichment chamber I and the balance pool II and is sealed by a sleeve structure. An outer sleeve is provided with a driving mechanism and a mobile digital stand. An inner sleeve is assembled with the cell exposure membrane box.

The driving mechanism of the exposure module III is located at a bottom of the cell exposure membrane box, is provided with a solenoid valve and a flow controller, provided with one or a combination of two of a micro pipeline axial flow fan and an air pump, and includes a moving track, a magnetic coil, and a magnetic levitation motor.

The cell exposure membrane box of the exposure module III may rotate along the moving track, and a rotating speed is adjustable.

The magnetic levitation motor of the exposure module III can drive the cell exposure membrane box to rotate to assess cell exposure of bioaerosols in air, so as to ensure reliable operation of a cell exposure process based on an in-vitro model.

The cell exposure membrane box of the exposure module III is supported by the 3D grid. The grid includes a microtube structure and a wireless sensor, for example, a pressure sensor. An outlet tube diameter of the microtube structure of the 3D grid is greater than or equal to 0.25 mm, and is clamped with a membrane block made of a porous material with a gradient bore diameter (a gray membrane in FIG. 1). A bore diameter is greater than or equal to 0.25 mm, and a bore diameter gradient is not lower than 0.1 mm/mm. The material is silicon dioxide, polytetrafluoroethylene, polystyrene, but is not limited to this.

The exposure module III collects data of concentration and particle size of biological particles in air according to the wireless sensor and a cell-based biosensor, adjusts a digital component for a rotating speed in real time, and has a function of recoding a moving trajectory of the enrichment device.

Generally, a cell exposure research method and use of a mobile centrifugal impact-based bioaerosol enrichment device according the present disclosure are characterized in that the bioaerosol enrichment device includes a centrifugal impact-based bioaerosol enrichment device main unit and a cell exposure system. The centrifugal impact-based bioaerosol enrichment device main unit includes an enrichment chamber, a balance pool, and an exposure module.

The cell exposure method for the bioaerosol enrichment device according to the present disclosure is characterized in that:

The mobile centrifugal impact-based bioaerosol enrichment device is based on a gas-liquid exposure principle, and adopts the cell exposure method including steps of real-time collection, intelligent adjustment, and dynamic control and exposure. A detail exposure step is as follows:

### S1. Real-Time Collection:

Air sequentially enters a heavy dust bin, a trace dust bin, and a microbial enrichment bin of the enrichment chamber for graded collection; an air outlet of the microbial enrichment bin is connected to an inlet in a bottom end of the balance pool of the bioaerosol enrichment device through a pipeline, a micro valve, and a wireless sensor; and after that, the air enters a throttle element and a pore plate through the filter mesh, the atomizer, and the electrostatic generator of the balance pool, and is sprayed into the exposure module through a spray nozzle, so as to ensure accuracy of collected data.

### S2. Intelligent Adjustment:

The cell exposure system includes a cell-based biosensor and a programmable controller, may be installed with a data processing unit and a memory, and can intelligently adjust exposure temperature, humidity, flow rate, and components of the air entering the exposure module according to instructions of the programmable controller, and dynamically control a cell exposure environment and store data. The mobile digital stand sets a cell exposure limit according to information such as concentration of bioaerosols in air collected by the wireless sensor in real time, inputs the cell exposure limit into the cell-based biosensor system, and issues instructions to adjust a cell exposure parameter of the exposure module III.

### S3. Dynamic Control:

One end of the 3D grid of the exposure module III is connected to the wireless sensor of the balance pool, and the other end is connected to the cell-based biosensor system. A cell-based biosensor wrapped with gel is quantitatively delivered to a surface of the membrane block made of the porous material through a metering pump, and information about cell growth, metabolism, images, and the like is collected and is transmitted back to the programmable controller and the data processing unit for recording, storing, and analyzing cell exposure data to realize dynamic control of the bioaerosols.

### S4. Exposure:

A control center of the cell exposure method is a cell-based biosensor. Exposure research is performed in the exposure module with a rotatable component. The exposure module is sealed through a silicone rubber element and a sleeve structure, and the driving mechanism, the cell exposure membrane box, and the mobile digital stand are mounted. The cell exposure membrane box is a rotatable exposure component, is supported by the 3D grid, and is clamped with the membrane block made of the porous material. A track, a magnet, and a magnetic levitation motor of the driving mechanism are arranged on an outer sleeve, and is connected to the cell exposure membrane box through a microtube of the 3D grid. The 3D grid includes near-end microtubes, far-end microtubes, and side-end microtubes. The near-end microtubes are connected to a liquid inlet pipe and a liquid outlet of the metering pump, the mobile digital stand, and the programmable controller; the far-end microtubes are connected to the membrane block made of the porous material; and part of the side-end microtubes extends out of the sleeve structure to connect the wireless sensor and the throttle valve, and the other part of the side-end microtubes is connected to the cell-based biosensor system. Cell exposure data is recorded.

In conclusion, the mobile centrifugal impact-based bioaerosol enrichment device and the cell exposure method and use thereof are simple and easy to implement, solve problems about enrichment and mobile monitoring of low concentration bioaerosols (including viruses, bacteria, fungi, and the like on particles) in air, and provide scientific data for bioaerosol spreading and risk control of crowd activity places such as transportation vehicles, livestock farms, and biological laboratories. Moreover, a bioaerosol exposure method is provided, which facilitates in-situ monitoring of cell exposure toxicity, and has broad application prospects and value.

Compared with the prior art, the present disclosure has the following beneficial effects.

1. The mobile centrifugal impact-based bioaerosol enrichment device according to the present disclosure is compact in structure, uses a non-contact “bioaerosol release source positioning” mobile measurement technology, improves the enrichment efficiency of the bioaerosols in the air, and has flexibility of multi-dimensional environmental monitoring in time and space.

2. The mobile centrifugal impact-based bioaerosol enrichment device according to the present disclosure uses a method for analyzing information about concentration of the bioaerosols in the air by using the cell-based biosensor system, achieves comprehensive cell exposure data, and improves accuracy of the accuracy of analyzing sources of microbial pollutions in the air.

3. A rotary cell exposure system connected to the balance pool is constructed, which solves a problem that it is difficult to continuously expose cells in real environmental pollutants, avoids uncertainty of single-cell analysis of single pollutants, and provides a feasible method and a new idea for cell exposure research of bioaerosols.

### BRIEF DESCRIPTION OF THE DRAWINGS

FIG. 1 shows schematic diagrams of a front view of a top view of a mobile centrifugal impact-based bioaerosol enrichment device according to the present disclosure;

FIG. 2 is a flowchart of cell exposure of a centrifugal impact-based bioaerosol enrichment device according to the present disclosure;

FIG. 3 is a schematic diagram of gradient bore diameters of a membrane block made of porous materials of an exposure module III according to the present disclosure;

FIG. 4 is a schematic structural diagram of a 3D grid and a microtube structure of an exposure module III according to the present disclosure;

FIG. 5 is a control schematic diagram of a cell-based biosensor system according to the present disclosure.

In the drawings: I-enrichment chamber; II-balance pool; III-exposure module; 1, heavy dust bin; 2, trace dust bin; 3, microbial enrichment bin; 4, atomizer; 5, membrane tube; 6, cell exposure membrane box; 7, membrane block; 8, electrostatic generator; 9, air outlet; 10, filter mesh sheet; 11, driving mechanism; 12, mobile digital stand; D1 to D4: micropore diameters of membrane block made of porous material; 13, 3D grid; 14, hydrogel coating; 15, microtube structure; 16, sensor; and 17, static mixer.

### DETAILED DESCRIPTION OF THE EMBODIMENTS

Content of the present disclosure is further described below with reference to specific embodiments, but is construed as a limitation to the present disclosure. If not specifically specified, technical means used in the embodiments are conventional means well-known to those skilled in the art. Unless otherwise specified, reagents, methods, and equipment used in the present disclosure are conventional reagents, methods, and equipment in this technical field.

### Embodiment

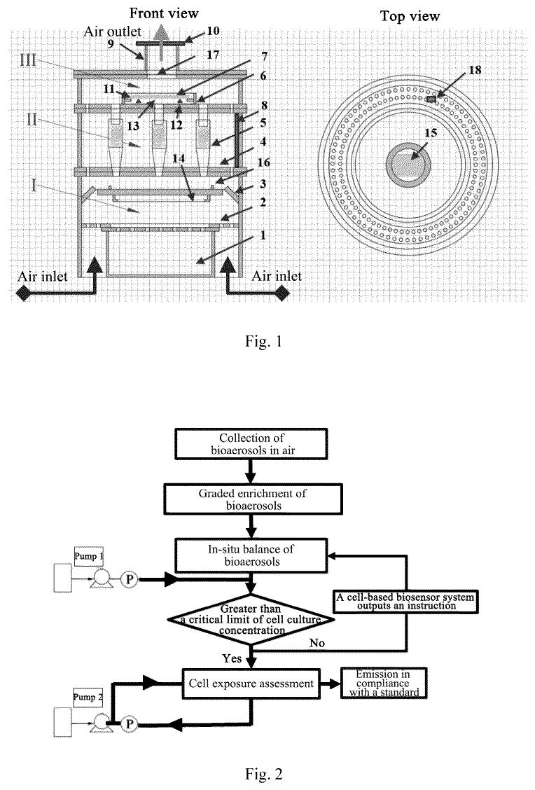

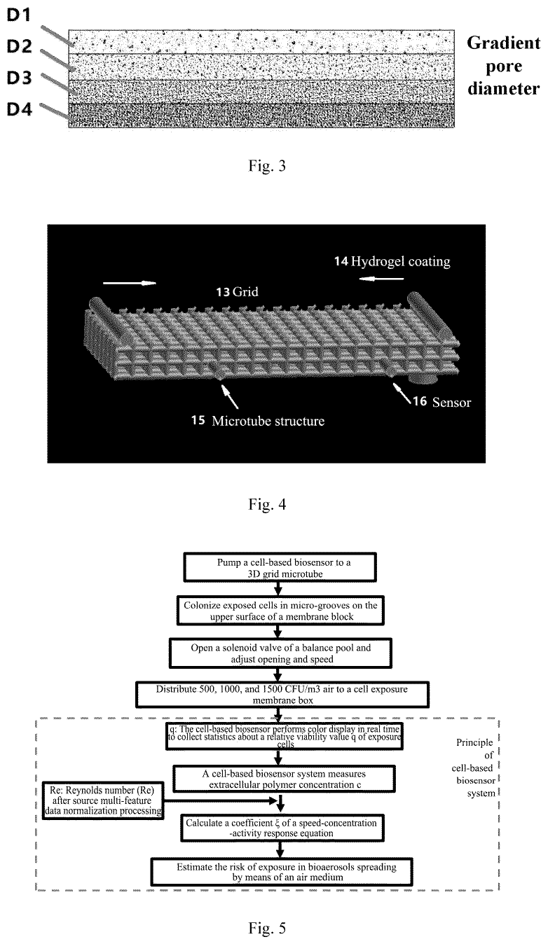

Referring to FIG. 1, FIG. 3, and FIG. 4, this embodiment provides a mobile centrifugal impact-based bioaerosol enrichment device. The enrichment device includes the following structures:

As shown in FIG. 1, the device includes an enrichment chamber I with grading chambers, a balance pool II, and an exposure module III. The enrichment chamber I includes a heavy dust bin 1, a trace dust bin 2, and a microbial enrichment bin 3. An inner surface of the microbial enrichment bin is coated with hydrogel 14. A bottom end of the balance pool II is connected to an air outlet 9 of the enrichment chamber I, and a CA filter mesh 10, an atomizer 4 that can generate microwave droplets of 0.5 to 50 μm, and a micro electrostatic generator 8 with an electrostatic voltage capable of being freely adjusted between 0 and 100 kv are mounted. The exposure module III is arranged below the enrichment chamber I and the balance pool II, and has a driving mechanism 11, a cell exposure membrane box 6, and a mobile digital stand 12. An inverted U-shaped levitation track, a neodymium-iron-boron U-shaped magnet, and a magnetic levitation direct-current motor are mounted at a bottom of the cell exposure membrane box 6 of the driving mechanism 11 to drive the cell exposure membrane box 6 to rotate. The cell exposure membrane box 6 is supported by a grid 13. The grid includes a capillary 3D printed microtube structure 15 and a PM, pressure, pressure difference, temperature, and humidity sensor 16. A polystyrene porous material membrane block 7 is clamped in the cell exposure membrane box 6.

FIG. 3 is a schematic diagram of gradient bore diameters of a membrane block made of a porous material of the exposure module III according to the present disclosure. Diameters D1>D2>D3<D4<10 μm, which ensures accuracy of monitoring concentration of bioaerosols in air. The mobile digital stand 12 of the exposure module III can adjust a speed according to collected data such as concentration and a particle size of particles in air, and record a moving trajectory of the enrichment device. The exposure module III is connected to an air outlet 9 of the balance pool II, and a static mixer 17 is mounted at the air outlet 9.

As shown in FIG. 4, the 3D grid and a microtube structure of the exposure module III of the present disclosure are in ingenious designs, which achieves an effect of uniformly distributing bioaerosols, reduces abnormal fluctuations of measurement data of a sensor connected to the mobile digital stand, facilitates carrying out cell exposure research in a real air environment, expands functions of a bioaerosol enrichment device in a field of cell exposure researches, and is beneficial to reducing the labor and time costs of repeated monitoring of the bioaerosols in the air.

A mobile centrifugal impact-based bioaerosol enrichment device and a cell exposure flowchart thereof according to the present disclosure are as follows:

1. Before monitoring, mobility of the enrichment device is detected first, a power supply of the driving mechanism 11 of the exposure module III is turned on, the cell exposure membrane box 6 is rotated, and whether various components of the exposure module III are dynamically balanced or sealed is detected by using a vibration analyzer, a dynamic balance instrument, and a pipeline air leakage detector. Subsequently, an adjusting capability of the balance pool II, whether the atomizer 4 and the electrostatic generator 8 are abnormal, and whether real-time control, data processing, and signal transmission of concentration of pollutants may be achieved are checked. Whether various bins of the enrichment chamber I may be communicated normally is observed, whether an atmospheric pollutant concentration monitoring sensor may be started normally is detected, then an air pump is started, so that the instruments start to operate, and it is determined that the device can continue to perform normal collection operation after the instruments are stabilized for 5 minutes. Next, the data stability of the enrichment device in collecting a standard sample, that is, a standard 500 CFU/m³ Lactobacillus gas sample, is tested, and a pressure is controlled between 0.1 and 20 kPa to observe that a fluctuation rate of the concentration of the bioaerosols is not greater than 10%. Finally, it is determined that an electric quantity of the device is sufficient, a temperature is at 0 to 50° C., and a humidity is at 10 to 90%, and valves of an air inlet and an air outlet 9 and a sterile cover of the enrichment device are opened. The mobile centrifugal impact-based bioaerosol enrichment device is worn on an arm of a testee to ensure comfort and stability of a wearing position of a wearer and prepare for sampling.

2. During sampling, a main power supply of the enrichment device is turned on, after the bioaerosol enrichment device is preheated for 3 to 5 minutes, an air inlet flow rate of the enrichment chamber I is set, valves of the heavy dust bin 1, the trace dust bin 2, and the microbial bin 3 of the enrichment chamber are opened, and a temperature and humidity sensor 16 and a GPS are started to collect data of the bioaerosols in the air and draw a concentration curve of the bioaerosols. The atomizer and a negative ion generator of the balance pool II are manually or automatically started periodically according to a programmed exposure threshold, a balance concentration of the bioaerosols in the air is controlled within a range of 50 to 5000 CFU/m³, and a solenoid valve of the balance pool II is adjusted when the device displays that a balanced concentration reaches a balance concentration. Next, a switch of an outlet pipe valve of the balance pool II is pressed down to deliver gas to the exposure module III, and a timer starts to time to simulate exposure of human nasopharyngeal epithelial cells. At this moment, the mobile digital stand 12 adjusts a rotating speed of the cell exposure membrane box according to the concentration of the bioaerosols in the air. In a case that the sensor detects that an exposure concentration of the cell exposure membrane box 6 is higher than an allowable cell limit 5000 CFU/m³, the device gives an alarm, and the outlet pipe valve of the balance pool II is closed temporarily, a log is created and is stored in a data processing unit and a memory chip, and sampling is ended.

3. After sampling is completed, first, the valve of the air outlet 9 provided with the static mixer is closed, the power supply of the driving mechanism 11 of the exposure module III is turned off, the cell exposure membrane box 6 is removed and is sent to a laboratory for retesting and emitting gas, and then, electronic and electrical components such as the heavy dust bin 1, the trace dust bin 2, the microbial bin 3, and the sensor 16 are removed, the balance pool II of the enrichment device is subjected to steam sterilization at 121° C., and the device is covered with a film for sealing under a sterile condition. A state of a programmable controller of the enrichment device is checked again, and stored data of the bioaerosols in the air is downloaded. Meanwhile, a system gas path, a filter mesh sheet 10, and the static mixer 17 are cleaned and replaced according to a biological pollution situation.

### Embodiment 1

The bioaerosol enrichment device includes an enrichment chamber I with grading chambers, a balance pool II, and an exposure module III. The enrichment chamber I is provided with a heavy dust bin 1, a trace dust bin 2, and a microbial enrichment bin 3. An inner surface of the microbial enrichment bin is coated with hydrogel 14. A bottom end of the balance pool II is connected to an air outlet 9 of the enrichment chamber I, and a CA filter mesh 10, an atomizer 4 that can generate microwave droplets, and a high-accuracy and high-stability +/−1200V electrostatic generator 8 are mounted. The exposure module III is arranged below the enrichment chamber I and the balance pool II, and includes a driving mechanism 11, a cell exposure membrane box 6, and a mobile digital stand 12. The driving mechanism 11 is provided with an O-shaped moving track and a magnetic levitation acoustic motor are mounted at a bottom of the cell exposure membrane box 6, and is connected to the air outlet of the balance pool II. The cell exposure membrane box 6 is rotatable, and is supported by a 3D grid 13. The grid includes a capillary microtube structure 15 and a PM, pressure, pressure difference, temperature, and humidity sensor 16. A polystyrene porous material membrane block 7 is clamped in the cell exposure membrane box 6. The mobile digital stand 12 adjusts a rotating speed of the cell exposure membrane box 6 according to the collected PM concentration of air.

The mobile centrifugal impact-based bioaerosol enrichment device according to the present disclosure may be applied to different scenarios, and a cell exposure research method is adjusted according to the scenarios.

This embodiment provides a mobile centrifugal impact-based bioaerosol enrichment device and use of a cell exposure method in indoor and outdoor areas. For a biochemical industry park, a bioaerosol collection and exposure method includes the following steps.

At S1: Sampling point setting: 25 grid sampling points were defined in a 1000 m² biochemical industry park, 4 occupational workers (2 males and 2 females) were selected at each sampling point, and a mobile centrifugal impact-based bioaerosol enrichment device according to the present disclosure was movably mounted to carry out cell exposure research.

At S2: Enrichment: After that, a power supply of the device was started, bioaerosols entered the heavy dust bin 1, the trace dust bin 2, and the microbial enrichment bin 3 in grades first under acting forces such as an inertial force and a centrifugal force through a hollow-out part of a mask of an enrichment chamber I with a wedge-shaped microstructure.

At S3: Hydrogel preparation and balance: 4.5 g of PVDF and 1.5 g of PSU were dissolved in 5 ml of polyethylene glycol-400, 15 ml of N-methyl pyrrolidone, and 5 ml of benzoic acid at 80° C. and were stirred for 2 hours until the PVDF and the PSU were dissolved to obtain a solution, an outer surface and an inner surface of the microbial enrichment bin 3 of the enrichment chamber I and a membrane tube 5 were coated with the solution, were immersed in a 25° C. water bath and 100 mg of ethanol solution of carotenoid ester, and were cured to form a hydrogel coating, where the coating was defined with a thickness of 5 mm, and had a papillary microstructure. The activity and concentration of the bioaerosols of the heavy dust bin 1 were measured at a temperature of 36.5 to 37.5° C., a humidity of the air emitted from the balance pool II was controlled to be 60 to 65 RH %, and PM2.5 concentration was lower than 10 μg/m³.

S4: Exposure: A rotating speed of the cell exposure membrane box was set as 6 to 9 r/min, a 50.25 cm² PES membrane block 7 after high-temperature and high-pressure sterilization treatment was arranged, an outer surface of the membrane block 7 was coated with 10 ml of cell tissue preservation solution, and (1 to 5)×10⁵/ml human esophageal epithelial cells after colonization and amplification culture were inoculated, where an inoculation amount was 50 μL; air emitted from the balance pool II and a cell-based biosensor were introduced below the membrane block, where the temperature was adjusted to 37.3° C., the humidity was adjusted to 28 RH %, and a flow rate was adjusted to 400 L/min; and exposure lasted for a working time of 8 hours.

At S5: Normalization processing of source feature data: The cell exposure membrane box and a programmable controller with data collection and storage functions were connected to a personal computer (PC) to perform data analysis to assess daily and hourly exposure risks of 100 occupational workers to bioaerosols online.

At S6: Gas emission in compliance with a standard: the emitted gas was treated by using an electrostatic generator 8 arranged at an air outlet 9, so that the gas was emitted in compliance with a standard, and the PM 2.5 concentration was lower than 1 μg/m³.

### Embodiment 2

As shown in FIG. 2, for exposure risk research in a public restroom with a high humidity, a high temperature, and a high bioaerosol concentration, a cell exposure method includes the following steps:

At S1: Fixed sampling point setting: A dry area and a wet area of a public toilet in a shopping mall were divided, one mobile centrifugal impact-based bioaerosol enrichment device according to the present disclosure was arranged at each of the dry areas and the wet areas of male and female public restrooms and galleries on a sixth floor, and exposure risks of the public restrooms on a rest day (Sunday) and working days (Monday to Saturday) were measured.

At S2: Real-time collection: Bioaerosol enrichment fixing devices were mounted in the male and female public restrooms and the galleries, aerosols in air entered a heavy dust bin 1, a trace dust bin 2, and a microbial enrichment bin 3 based on a centrifugal impact method, and a UV-LED strip was mounted at a bottom of the heavy dust bin 1, where a wavelength was 254 nm, and a wavelength accuracy was +/−1 nm.

At S3: Intelligent adjustment: to-be-processed data collected by a wireless concentration sensor and a wireless temperature sensor was linearly transformed into a normalized data format through a statistical method (a mathematical method), and a dimensional influence attached to the data was reduced, so that different feature data have the same scale, and have comparability. Source multi-feature data was normalized by using a min-max normalization method, and data transformation was performed by using the following formula:

$${X}^{′} = \frac{x - {x}_{min}}{{x}_{ma} - {x}_{min}} \qquad formula 1$$

where min represents a minimum value of multi-feature data, max represents a maximum value of the multi-feature data, and x represents a specific value in metadata. Normalized data was input into a cell-based biosensor system, and opening and speed of the solenoid valve of the balance pool were adjusted according to a principle in FIG. 4.

At S4: Dynamic control: Hydrogel was prepared, that is, 2.5 g of PES was dissolved in 15 ml of dichloromethane at 80° C. with stirring until fully dissolved. The solution was then sprayed through a four-axis nozzle into a 20° C. water bath containing 10 mL of droplets formed from 100 mg of polycarotenoid acrylate dissolved in 1 mL of ethanol solution. The solution and the droplets formed the gel, and then the gel was applied in an array onto a top plate of a microbial enrichment bin 3 of the enrichment chamber I. A colony count concentration of the microbial enrichment bin 3 was calculated by a colony count method, a temperature was controlled at 36.5 to 37.5° C., and a physical and chemical environment of multi-concentration bioaerosols in air with a cell culture limit greater than or equal to 50 CFU/m³ was dynamically adjusted.

At S5: Cell exposure: A rotating speed of the cell exposure membrane box 6 was set as 20 r/min, a 12.5 cm² membrane block 7 after high-temperature and high-pressure sterilization treatment was arranged, a surface of the membrane block 7 was coated with 2 ml of cell tissue preservation solution, (1 to 5)×10⁶/ml human alveolar epithelial cells after colonization and amplification culture were inoculated, where an inoculation amount was 100 μL; and air with the humidity of 80 to 95 RH %, PM 2.5 concentration of 90 to 110 μg/m³, a temperature of 36.5° C., and a flow rate of 600 ml/min emitted from the balance pool II was inflated below a lower end of the membrane block to assess exposure risks of the public restrooms on a rest day (Sunday) and working days (Monday to Saturday).

At S6: Safe emission: a 25 Kv electrostatic generator and a medical atomizer were arranged at the air outlet 9, atomized droplets were in normal distribution, a diameter distribution range was 0.1 to 500 μm, an atomization flow rate was 2 ml/min, and an atomization speed was 0.5 m/s. Gas was emitted from the air outlet in compliance with a standard, a PM 2.5 concentration of the emitted gas was lower than 1 μg/m³, and a culturable microbe concentration was not higher than 1 CFU/m³.

### Embodiment 3

As shown in FIG. 5, for a biological laboratory scenario with low concentration of bioaerosols in air, a bioaerosol enrichment and cell exposure method includes the following steps:

At S1: Mobile sampling point setting: 3×3 grids were divided in a biosafety level II laboratory, and 9 sampling points were arranged, three rings spaced 1.5 meters apart were divided out of a biological laboratory, 18 sampling points in total were arranged at every 60°, a laboratory worker was selected, a bioaerosol enrichment device according to the present disclosure was worn on the left upper arm of the laboratory worker, bioaerosol exposure data of the laboratory worker during working was collected every other 4 hours, and a program of the enrichment device was set, so that a rotating speed of a cell exposure membrane box 6 was stabilized at 1.1 to 10 r/min, and an exposure research time was 72 hours.

At S2: Enrichment: Air was connected to an air inlet at a lower end of the enrichment device, and the air entered a balance pool II after passing through an enrichment chamber I. A membrane tube 5 in the enrichment device was washed with normal saline to collect bioaerosols for 3 to 5 times, and an outer surface of the tube was wound with hydrogel filaments: 5 g of PES was dissolved in 2 ml of tween-60 and 20 ml of dimethylformamide at 73 to 75° C. with stirring until fully dissolved. The solution was then sprayed through a coaxial double-row hexagonal tube spinnerets into a 4° C. cold water bath containing 100 mg of carotenoid ester, a fluorescence reagent, and 10 ml of an ethanol solution with 0.5 to 1.5 wt % of yeast extract. Winding was performed after solidification. Then, biochemical indexes such as bioaerosol concentration was calculated by a fluorescence method. After a cell culture concentration limit was greater than a critical limit, the air was re-pumped into the balance pool II to increase a winding density of the hydrogel on the surface of the membrane tube, the membrane tube was washed twice by using the atomizer with a Dulbecco's phosphate-buffered saline (DPBS) solution containing 0.02% ethylene diamine tetraacetic acid (EDTA), and then gas was emitted by using a riser of the membrane tube and was delivered to an exposure module III.

At S3: Cell exposure: A rotating speed of the cell exposure membrane box 6 was set as 6 r/min, a 25 cm² membrane block 7 after high-temperature and high-pressure sterilization treatment was arranged, a surface of the membrane block 7 was coated with 2 ml of cell tissue fixative, 100 μl of (1 to 1.5)×10⁵/ml human nasal mucosa epithelial cells after colonization and amplification culture were inoculated, and air with a temperature of 13 to 14, a humidity of 28 to 32% RH, a PM 2.5 concentration of 40 to 60 μg/m³, a PM 2.5 concentration of 200 to 220 μg/m³, a PM 10 concentration of 220 to 320 μg/m³, a flow rate of 500 ml/min, and bioaerosol concentrations of 500, 1000, and 1500 CFU/m³ emitted from the balance pool II was injected into a side wall of the membrane block. Cell exposure risks of the bioaerosols in and out of the biological laboratory within 24 hours were assessed, and the cell exposure membrane box might be replaced timely according to an exposure situation.

At S4: Construction of cell-based biosensor system: small wells were corroded in micro-grooves of a liner of the membrane block 7 made of a silica porous material, a cell-based biosensor was pumped by a microtube of a 3D grid, were filled into the grooves, and covered a hydrogel culture medium. The cell-based biosensor was scanned by using an LED, and generation of electron-hole pairs was excited through pulse laser. Parameters such as extracellular pH value after exposure cells exposed the bioaerosols in air were analyzed by a programmable controller connected to the other end of the microtube, as shown in FIG. 5. When a caused signal fluctuation exceeded a threshold stimulation point, the signal fluctuation was fed back to the balance pool II, a data processing unit, and a memory to readjust the concentration of the bioaerosols in the balance pool II. In a case that the caused signal fluctuation did not exceed the threshold stimulation point, inherent cell exposure parameters were maintained. The cell-based biosensor performed color display in real time to collect statistics about a relative viability value q of exposure cells, and then the programmable controller combined a Reynolds number Re and an extracellular polymer concentration c output by a wireless sensor, and a PC calculated to obtain a coefficient § of a speed-concentration-activity response equation constructed based on a biological laboratory scenario of laboratory worker activities. Finally, the risk of exposure in bioaerosols spreading by means of an air medium was estimated and the data was input into a storage chip.

S5: Safe emission: The bioaerosol-containing air was emitted from the air outlet 9 in compliance with a standard after passing through the 25 KV electrostatic generator 8, a high efficiency particulate air (HEPA) filter element, and the atomizer 4 for atomizing normal saline that contains penicillin and/or streptomycin sulfate, and a static mixer 17, where the PM 2.5 concentration was lower than 1 μg/m³, and a culturable microbe concentration was not higher than 1 CFU/m³.

The above descriptions and accompanying drawings fully demonstrate the embodiments of the present disclosure, so that those skilled in the art can practice them. Other embodiments may include changes in device structure, computer programs, electronic and electrical systems, dynamic enrichment processes, and others changes. The embodiments only represent possible changes, unless otherwise specified, individual components and functions are optional, and operation sequences are changeable. Parts of features of some embodiments may be included in or replace part features of other embodiments. Moreover, words used in this application are only used for describing the embodiments rather than limiting claims of the present disclosure.

In the embodiments disclosed herein, the disclosed method and product (including, but not limited to, a device, equipment, and the like) may be implemented in other manners. For example, the device embodiment described above is only schematic, for example, division of a sampling grid may only be division of crowd activity functional areas. There may be additional sampling grid division manner in an actual implementation, for example, assembly of various bins of the bioaerosol enrichment chamber may be combination or integration to another system. Alternatively, some features may be neglected or not executed. In addition, the displayed or discussed mutual couplings or direct couplings or communication connections between electronic components may be indirect couplings or communication connections through some digital interfaces, devices, or systems, or may be in electronic, mechanical, or other forms. Some or all of the unit modules may be selected according to implement the present disclosure according to actual needs. In addition, various modules in the embodiments of the present disclosure may be integrated in one system or may exist in combined with a plurality of systems, or a single module may operate independently.

The foregoing embodiments are preferred implementations of the present disclosure. However, implementations of the present disclosure are not limited by the foregoing embodiments. Any other changes, medications, replacements, combinations, and simplifications made without departing from the spiritual essence and principles of the present disclosure are all equivalent replacement manners, which are all included in the scope of protection of the present disclosure.

1. A mobile centrifugal impact-based bioaerosol enrichment device, comprising a mobile centrifugal impact-based bioaerosol enrichment device main unit and a cell-based biosensor system, wherein an air inlet of the enrichment device is communicated with air and comprises an enrichment chamber, a balance pool, an exposure module, and the cell-based biosensor system; through the air is communicated with the air inlet of the enrichment device, passes through a flexible pipeline, a micro valve, and a wireless sensor after sequentially passing through a heavy dust bin, a trace dust bin, and a microbial enrichment bin, and is connected to a bottom inlet of the balance pool; a concentration of the bioaerosols in the air is adjusted through a filter mesh, an atomizer, and an electrostatic generator, and then the air is connected to the exposure module with a rubber piece and a sleeve sealing structure through a throttle element and an adjustment valve; a driving mechanism and a mobile digital stand together drive a cell exposure membrane box to rotate to expose cells; the cell exposure membrane box is supported by a 3D grid; microtubes of the 3D grid penetrate out of the sleeve sealing structure and is connected to the cell-based biosensor system to dynamically adjust exposure conditions; and finally, the air is emitted from an air outlet in compliance with a standard.

2. The mobile centrifugal impact-based bioaerosol enrichment device according to claim 1, wherein the microtubes of the 3D grid of the exposure module comprise near-end microtubes, far-end microtubes, and side-end microtubes; the near-end microtubes are connected to a liquid inlet tube and a liquid outlet of a metering pump, the mobile digital stand, and a programmable controller; the far-end microtubes are connected to a membrane block made of a porous material; the side-end microtubes extend out of a sleeve structure to connect the wireless sensor and a throttle valve; and a diameter of an outlet of the side-end microtube is greater than or equal to 0.25 mm.

3. A cell exposure research method, wherein steps of real-time collection, intelligent adjustment, and dynamic control are used to drive a cell exposure membrane box to rotate along a moving track to perform cell exposure based on a gas-liquid exposure principle by using the mobile centrifugal impact-based bioaerosol enrichment device according to claim 1.

4. The cell exposure research method according to claim 3, wherein a balance pool is provided with a valve with adjustable opening to delivery air with different concentrations of bioaerosols in different areas and distribute the air to an exposure module.

5. The cell exposure research method according to claim 3, wherein one end of the side-end microtube of the 3D grid is connected to the wireless sensor, and the other is connected to the cell-based biosensor; and a cell-based biosensor a pore size of a pore plate of a throttle element of a balance pool through a data processing unit, and adjusting opening of a micro valve on a flexible pipeline.

6. The cell exposure research method according to claim 3, wherein the cell-based biosensor system comprises a cell-based biosensor and a programmable controller; the cell-based biosensor is configured to measure cell exposure toxicity of bioaerosols in air; the programmable controller intelligently adjusts temperature, humidity, and pollution gas compositions and concentrations of exposure by issuing instructions to a balance pool.

7. The cell exposure research method according to claim 3, wherein a wireless sensor is capable of monitoring the concentration of bioaerosols in air in real time, inputting the concentration into a programmable controller, dynamically controlling a rotating speed of the cell exposure membrane box, and recording a change trend of cell exposure data according to a programmed cell exposure limit.

8. The cell exposure research method according to claim 3, wherein microtubes of a 3D grid and the metering pump of the cell exposure membrane box are capable of delivering the cell-based biosensor wrapped with gel to an upper surface of the membrane block and collecting information about cell growth, metabolism, and images, the like, and transmitting the information back to a programmable controller for recording and storing data, and cell exposure data is analyzed by a data processing unit.

9. The cell exposure research method according to claim 3, wherein bioaerosol-containing air of an exposure module is introduced into a static mixer coated with gel through a tree-like pipeline, wet deposition is performed on bioaerosols in air by using atomized droplets sprayed by a throttle nozzle, and the air is safely emitted from an air outlet after the concentration of the bioaerosols is reduced.
